(19) United States
(12) Patent Application Publication
(10) Pub. No.: US 2013/0310831 A1
(43) Pub. Date: Nov. 21, 2013

(54) JAWED ENDOSCOPIC INSTRUMENT FOR ELECTROSURGERY

(71) Applicants: VYSOKA SKOLA BANSKA-TECHNICKA UNIVERZITA OSTRAVA, (US); CESKE VYSOKE UCENI TECHNICKE V PRAZE, FAKULTA BIOMEDICINSKEHO INZENYRSTVI, (US)

(72) Inventors: Jan GREPL, Cervenka (CZ); Marek PENHAKER, Ostrava (CZ); Petr KOVÁR, Ostrava (CZ); Milada HLAVÁCKOVÁ, Ostrava -Hrabuvka (CZ); Pavel SMRCKA, Praha (CZ); Karel HÁNA, Ricany u Prahy (CZ); Jan KASPAR, Jicin (CZ)

(73) Assignees: CESKE VYSOKE UCENI TECHNICKE V PRAZE, FAKULTA BIOMEDICINSKEHO INZENYRSTVI, Kladno (CZ); VYSOKA SKOLA BANSKA-TECHNICKA UNIVERZITA OSTRAVA, Ostrava - Poruba (CZ)

(21) Appl. No.: 13/893,098

(22) Filed: May 13, 2013

(30) Foreign Application Priority Data
May 16, 2012 (CZ) PV2012-323

(51) Int. Cl. A61B 18/14

(52) U.S. Cl. CPC A61B 18/1445
USPC 606/41

(57) ABSTRACT

A jawed endoscopic instrument for electrosurgery is designed as a two-section tool, where one section includes a supporting rod with a fixed jaw portion, the other section a movable jaw portion attached to the supporting rod with the fixed jaw portion with a pin joint. A magnet is inserted in the movable jaw portion and covered with a clamped electrode, and another magnet mounted on a pull is inserted into the cavity of the supporting rod with the fixed jaw portion and covered with another electrode. When the pull moves, the movable jaw portion will be opened or closed relative to the supporting rod with the fixed jaw portion due to a change in the magnetic field between the magnets.

### BACKGROUND AND SUMMARY

The invention relates to instruments used in surgery and gynaecology as well as in electrical engineering and biomedical engineering.

The instruments used at present are endoscopic instruments based on a mechanical principle or electrosurgical instruments based on the principle of unipolar coagulation of the tissue to the electrode at the end of the endoscopic instrument, or as bipolar between two electrodes on an instrument without movable parts. There are also bipolar electrosurgical scissors with electrodes in the jaw portions, operated by a pull from the doctor's handle.

The present invention concerns a jawed endoscopic instruments for electrosurgery, comprising a fixed section to which at least two jaw portions are attached, at least one of them movable. The fixed section of the instrument is a supporting rod with a fixed jaw portion and the movable jaw portion is attached to the supporting rod with the fixed jaw portion by means of a pin joint.

A magnet is inserted in each movable jaw portion, covered with an electrode, and another magnet is fixed to the pull. The pull with the magnet is movably inserted in a cavity in the supporting rod with the fixed jaw portion, whose end is covered with a fixed another electrode.

Importantly, the electrodes are connected to supply wires, leading to the end of the instrument through the supporting rod with the fixed jaw portion.

And it is essential that when the pull is moved, the jaw portions are opened or closed due to a change in the distribution of magnetic forces between the magnets in the jaw portions.

### BRIEF DESCRIPTION OF THE DRAWINGS

The invention is explained in more detail in the accompanying drawings, where

FIG. 1 is a view of the device in its unfolded state.

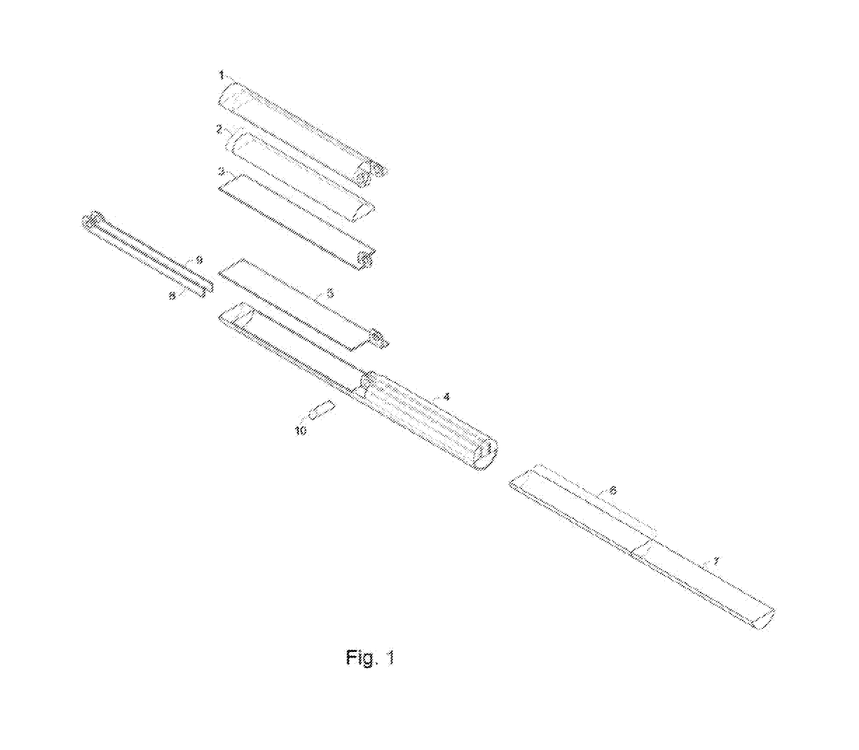

FIG. 2a) shows an overall view of the device in its basic position and

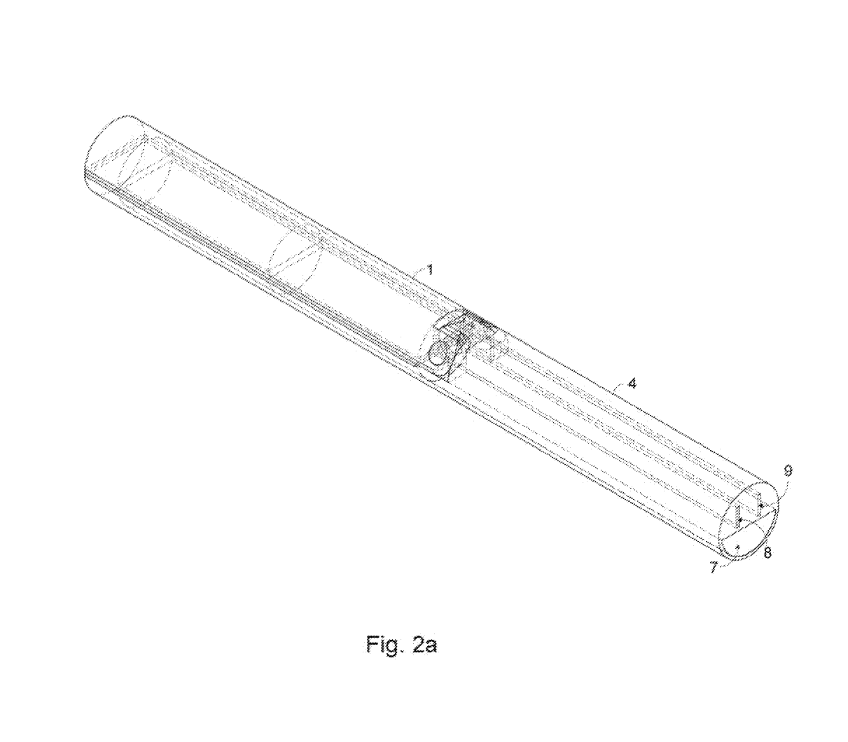

FIG. 2b) is a general view of the device in its opened position.

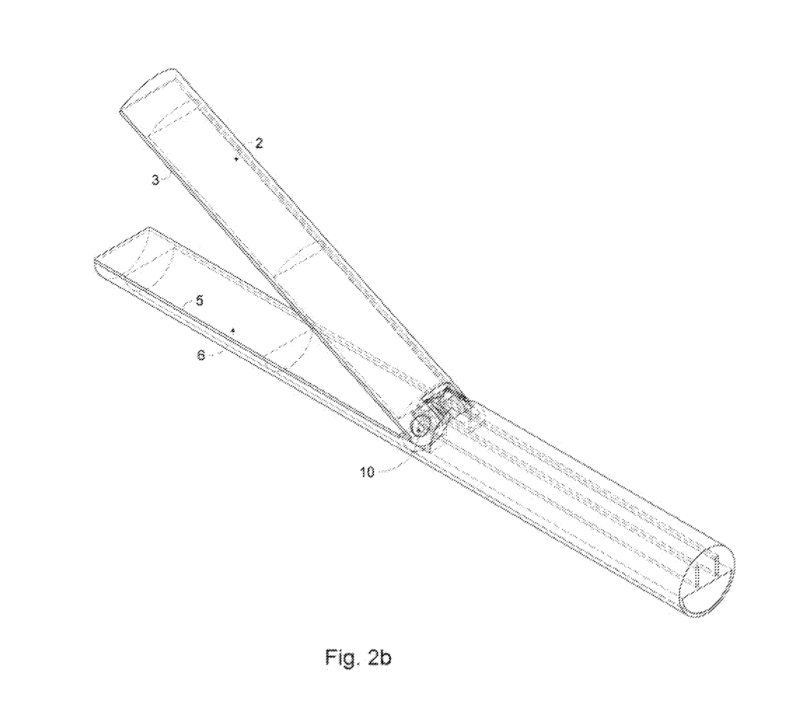

### DETAILED DESCRIPTION

This instrument is designed as a two-section tool, one section comprising a supporting rod 4 with a fixed jaw portion, the other section a movable jaw portion 1 attached to the supporting rod 4 with the fixed jaw portion with a pin joint 10,

A magnet 2 is inserted in the movable jaw portion 1 and covered with a clamped electrode 3. Another magnet 6 mounted on a pull 7 is inserted into the cavity of the supporting rod 4 with the fixed jaw portion, covered with another electrode 5. It is possible to move the pull 7 and another magnet 6 fixed to it. Another electrode 5 is connected to a supply wire 9 and the electrode 3 is connected to another supply wire 8. Both supply wires 9, 8 lead to the end of the instrument.

When the pull 7 moves, the jaw portions will be opened or closed due to a change in the distribution of magnetic forces between the magnets 2, 6 placed in the jaw portions.

The invention can be used in surgery, gynaecology, electrical engineering, biomedical engineering.

What is claimed is:

1. A jawed endoscopic instrument for electrosurgery, comprising:
a fixed section comprising a supporting rod with a fixed jaw portion;
at least one movable jaw portion movably attached to the supporting a rod via a pin joint;
a first magnet disposed in each movable jaw portion and covered with an electrode; and
a unit comprising a second magnet mounted on a pull, the unit being movably insertable in a cavity of the supporting rod and the second magnet being covered with a fixed electrode.

2. A jawed endoscopic instrument for electrosurgery as in claim 1, wherein the fixed electrode is connected to a first supply wire and the electrode is connected to a second supply wire, and the first and second supply wires lead to an end of the instrument through the fixed section.

3. A jawed endoscopic instrument for electrosurgery as in claim 2, wherein movement of the pull opens and closes the jaw portions due to a change in distribution of magnetic forces between the first and second magnets in the jaw portions.

4. A jawed endoscopic instrument for electrosurgery, as in claim 1, wherein movement of the pull opens and closes the jaw portions due to a change in distribution of magnetic forces between the first and second magnets in the jaw portions.
